(19) United States
(12) Patent Application Publication
(10) Pub. No.: US 2017/0320794 A1
(43) Pub. Date: Nov. 9, 2017

(54) PROCESS FOR PRODUCING BENZENE AND LPG2

(71) Applicant: SABIC Global Technologies B.V., Bergen op Zoom (NL)

(72) Inventor: Thomas Hubertus Maria Housmans, Geleen (NL)

(21) Appl. No.: 15/319,488

(22) PCT Filed: Jun. 1, 2015
(86) PCT No.: PCT/EP2015/062077

§ 371 Date: Dec. 16, 2016

(30) Foreign Application Priority Data
Jun. 17, 2014 (EP) 14172697.6

(51) Int. Cl. C07C 2/86; C07C 4/18; C10G 69/12; C10G 47/16; C10G 29/20

(52) U.S. Cl. CPC C07C 2/864; C10G 47/16; C10G 29/205; C10G 69/123; C07C 4/18; C07C 2529/08; C10G 2400/30

(57) ABSTRACT

The invention is directed to a process for producing benzene and LPG comprising the steps of: (a) reacting a source feed stream comprising monoaromatic compounds of formula (I), wherein R1-R5 are the same or different and are chosen from hydrogen or a linear alkyl group of 1-10 carbon atoms, and methanol in an alkylation reactor comprising a basic catalyst to obtain an alkylation product stream and subsequently (b) contacting the alkylation product stream in the presence of hydrogen in a hydrocracking reactor with a hydrocracking catalyst comprising 0.01-1 wt-% hydrogenation metal in relation to the total catalyst weight and a zeolite having a pore size of 5-8 Å and a silica (SiO2) to alumina (Al2O3) molar ratio of 5-200 to produce a hydrocracking product stream comprising benzene and LPG under process conditions including a temperature of 425-580° C., a pressure of 300-5000 kPa gauge and a Weight Hourly Space Velocity of 0.1-15 h⁻¹.

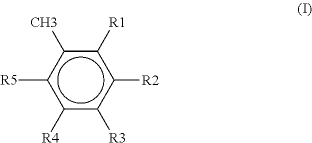

The invention is directed to a process for producing benzene and LPG.

Aromatics have a wide variety of applications in the petrochemical and chemical industries. These are important raw materials for many intermediates of commodity petrochemicals and valuable fine chemicals, such as monomers for polyesters, engineering plastics, intermediates for detergents, pharmaceuticals, agricultural products and explosives. Among them, benzene, toluene and xylene (BTX) are the three basic materials for most intermediates of aromatic derivatives.

About 70% of the global production of benzene is by extraction from either reformate or pyrolysis gasoline (pygas). Reformate is formed in the catalytic reforming of naphtha, a technology primarily directed at the production of high octane gasoline components. Pygas is a liquid byproduct formed in the production of olefins by steam cracking liquid feeds such as naphtha or gas oil. Extraction from reformate and pygas are the most economical sources of benzene. The composition of BTX depends on the source. Pygas is typically rich in benzene, whereas xylenes and toluene are the main components of reformate.

WO2013/182534 discloses a process for producing BTX from a C5-C12 hydrocarbon mixture using a hydrocracking/hydrodesulphurisation catalyst. According to WO2013/182534, the process results in a mixture comprising substantially no co-boilers of BTX, thus chemical grade BTX can easily be obtained.

While the process of WO2013/182534 advantageously limits the amount of BTX co-boilers in the obtained mixture, control of the ratio between benzene, toluene and xylene in the obtained mixture is limited. In some cases, it is preferred to obtain more benzene than other aromatics such as toluene and xylene.

It would therefore be desirable to provide a process for converting a C5-C12 hydrocarbon feedstream which results in an increased yield of benzene compared to known processes.

This object is achieved by a process for producing benzene and LPG comprising the steps of:

(a) reacting a source feed stream comprising monoaromatic compounds of formula (I),

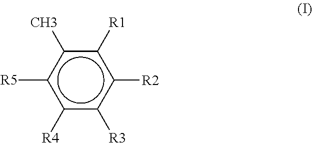

wherein R1-R5 are the same or different and are chosen from hydrogen or a linear alkyl group of 1-10 carbon atoms,

and methanol in an alkylation reactor comprising a basic catalyst to obtain an alkylation product stream and subsequently

(b) contacting the alkylation product stream in the presence of hydrogen in a hydrocracking reactor with a hydrocracking catalyst comprising 0.01-1 wt-% hydrogenation metal in relation to the total catalyst weight and a zeolite having a pore size of 5-8 Å and a silica (SiO2) to alumina

(Al2O3) molar ratio of 5-200 to produce a hydrocracking product stream comprising benzene and LPG under process conditions including a temperature of 425-580° C., a pressure of 300-5000 kPa gauge and a Weight Hourly Space Velocity of 0.1-15 h⁻¹.

The present inventors realized that a hydrocracking process results in a large portion of monoaromatic compounds such as toluene and xylene not being converted to benzene while compounds such as ethylbenzene are converted to benzene. The present invention is based on the realization that the benzene yield can be increased by increasing the amount of ethylbenzene or other compounds which would be converted into benzene by hydrocracking before subjecting a stream to hydrocracking.

In step (a) of the process of the invention, the methyl group of the monoaromatic compounds reacts with methanol and is converted to vinyl group or ethyl group. For example, toluene is converted to styrene or ethylbenzene. In the hydrocracking step (b), the ethyl group and any other alkyl group of the aromatic compounds leave the benzene ring and benzene is obtained. Hence, the monoaromatic compounds with one or more methyl groups as represented by formula (I) are converted to benzene in two steps according to the present invention.

### Source Feed Stream

The source feed stream is a hydrocarbon feed stream comprising at least monoaromatic compounds. The source feed stream can be chosen from a wide variety of hydrocarbon feed streams.

Preferably, the source feed stream comprises C5-C12 hydrocarbons. Preferably, the source feed stream comprises at least 40 wt %, more preferably at least 45 wt %, most preferably at least 50 wt % of the C5-C12 hydrocarbons.

The source feed stream may e.g. be chosen from pyrolysis gasoline, straight run naphtha, light coker naphtha and coke oven light oil or mixtures thereof.

The source feed stream may also comprise a stream comprising the alkyl monoaromatic compounds, wherein the stream is separated from the hydrocracking product stream. The source feed stream may also be a stream comprising the alkyl monoaromatic compounds, wherein the stream is separated from the hydrocracking product stream.

### Monoaromatic Compounds

The monoaromatic compounds are monoaromatic compounds of formula (I), wherein R¹-R⁵ are the same or different and are chosen from hydrogen or a linear alkyl group of 1-10 carbon atoms.

The compounds comprise one benzene ring and at least one methyl group. The monoaromatic compounds can comprise C7-57 compounds, preferably, C7-C20 compounds, more preferably C7-C12 compounds. R¹-R⁵ are preferably hydrogen or C1-C2 alkyl group. Examples of the monoaromatic compounds are toluene, xylene, cumene, 1,2,3-trimethyl benzene, 1,3,5-trimethyl benzene, p-cymene, 1-methyl-4-pentylbenzene, 1-methyl-4-ethylbenzene and 1,2-dimethyl-3-propylbenzene. The monoaromatic compound preferably comprises toluene, xylene and/or 1,3,5-trimethyl benzene.

### Alkylation Reactor

The source feed stream is reacted with methanol in the alkylation reactor in step (a) to produce an alkylation product stream. The methyl group(s) of the monoaromatic compounds react with methanol. The conditions and catalyst suitable for methanol alkylation is well-known and is described e.g. in A. E. Palomares, et. al., J. Catal., 1998 (180) 56.

The alkylation reactor comprises a basic catalyst, preferably a Bronsted basic zeolite catalyst. More preferably, the alkylation reactor comprises an X or Y zeolite catalyst modified with B, P, Na, K, Cs or Rb or mixtures thereof.

In the alkylation reactor the ratio between methanol and the monoaromatic compound preferably is between 1:1 and 1:20, more preferably between 1:1.5 and 1:15, most preferably between 1:2 and 1:10.

The temperature in the alkylation reactor preferably is between 100 and 1000° C., more preferably between 200 and 700° C., more preferably between 300 and 600° C. and most preferably between 350 and 440° C.

Ideally, all methyl groups in the monoaromatic compounds are converted to vinyl or ethyl group in step (a). Preferably, at least 40%, at least 50%, at least 60% or at least 70% of the methyl groups in the monoaromatic compounds are converted to vinyl or ethyl group in step (a).

Toluene is converted to styrene or ethylbenzene in step (a). Styrene and ethylbenzene are converted to benzene by hydrocracking step (b). Accordingly, the proportion of toluene is low in the alkylation product stream. Preferably, the proportion of toluene with respect to the total amount of toluene, styrene and ethylbenzene in the alkylation product stream is at most 50 wt %, at most 40 wt %, at most 30 wt %, at most 20 wt % or at most 10 wt %.

Xylene is converted to ethyl methyl benzene, methyl vinyl benzene, diethyl benzene, ethyl vinyl benzene or divinyl benzene. Styrene, ethylbenzene, diethyl benzene, ethyl vinyl benzene, divinyl benzene are converted to benzene by hydrocracking step (b). Ethyl methyl benzene and methyl vinyl benzene are converted to toluene instead of benzene by hydrocracking step (b). Accordingly, the proportion of xylene, ethyl methyl benzene and methly vinyl benzene is low in the alkylation product stream. Preferably, the proportion of the total of xylene, ethyl methyl benzene and methly vinyl benzene with respect to the total amount of xylene, ethyl methyl benzene, methly vinyl benzene, diethyl benzene, ethyl vinyl benzene and divinyl benzene is at most 50 wt %, at most 40 wt %, at most 30 wt %, at most 20 wt % or at most 10 wt %.

### Hydrocracking Reactor

The alkylation product stream is subsequently contacted with hydrogen in a hydrocracking reactor. The hydrocracking reactor is a so-called mild hydrocracking reactor containing a hydrocracking catalyst. The obtained hydrocracking product stream is advantageously substantially free from non-aromatic C6+hydrocarbons due to the catalyst and the conditions employed. Hence, according to the present invention, chemical grade benzene can easily be separated from the hydrocracking product stream product stream.

The advantageous effects of the hydrocracking step are obtained by strategically selecting the hydrocracking catalyst in combination with the hydrocracking conditions.

Hydrocracking is performed under process conditions including a temperature of 425-580° C., a pressure of 300-5000 kPa gauge and a Weight Hourly Space Velocity of 0.1-15 h⁻¹. By combining a hydrocracking catalyst having a relatively strong acid function (e.g. by selecting a catalyst comprising a zeolite having a pore size of 5-8 Å and a silica (SiO₂) to alumina (Al₂O₃) molar ratio of 5-200) and a relatively strong hydrogenation activity (e.g. by selecting a catalyst comprising 0.01-1 wt-% hydrogenation metal) with process conditions comprising a relatively high process temperature (e.g. by selecting a temperature of 425-580° C.), chemical grade BTX and LPG can be produced from the alkylation product stream.

Preferably, the hydrocracking of the feed stream is performed at a pressure of 300-5000 kPa gauge, more preferably at a pressure of 600-3000 kPa gauge, particularly preferably at a pressure of 1000-2000 kPa gauge and most preferably at a pressure of 1200-1600 kPa gauge. By increasing reactor pressure, conversion of C5+non-aromatics can be increased, but also increases the yield of methane and the hydrogenation of aromatic rings to cyclohexane species which can be cracked to LPG species. This results in a reduction in aromatic yield as the pressure is increased and, as some cyclohexane and its isomer methylcyclopentane, are not fully hydrocracked, there is an optimum in the purity of the resultant benzene at a pressure of 1200-1600 kPa.

Preferably, the hydrocracking of the feed stream is performed at a Weight Hourly Space Velocity (WHSV) of 0.1-15 h⁻¹, more preferably at a Weight Hourly Space Velocity of 1-10 h⁻¹ and most preferably at a Weight Hourly Space Velocity of 2-9 h⁻¹. When the space velocity is too high, not all BTX co-boiling paraffin components are hydrocracked, so it will not be possible to achieve BTX specification by simple distillation of the reactor product. At too low space velocity the yield of methane rises at the expense of propane and butane. By selecting the optimal Weight Hourly Space Velocity, it was surprisingly found that sufficiently complete reaction of the benzene co-boilers is achieved to produce on spec BTX without the need for a liquid recycle.

Accordingly, preferred hydrocracking conditions thus include a temperature of 425-580° C., a pressure of 300-5000 kPa gauge and a Weight Hourly Space Velocity of 0.1-15 h⁻¹. More preferred hydrocracking conditions include a temperature of 450-550° C., a pressure of 600-3000 kPa gauge and a Weight Hourly Space Velocity of 1-10 h⁻¹. Particularly preferred hydrocracking conditions include a temperature of 450-550° C., a pressure of 1000-2000 kPa gauge and a Weight Hourly Space Velocity of 2-9 h⁻¹.

The hydrocracking step is performed in the presence of an excess amount of hydrogen in the reaction mixture. This means that a more than stoichiometric amount of hydrogen is present in the reaction mixture that is subjected to hydrocracking. Preferably, the molar ratio of hydrogen to hydrocarbon species (H₂/HC molar ratio) in the reactor feed is between 1:1 and 4:1, preferably between 1:1 and 3:1 and most preferably between 1:1 and 2:1. A higher benzene purity in the product stream can be obtained by selecting a relatively low H₂/HC molar ratio. In this context the term “hydrocarbon species” means all hydrocarbon molecules present in the reactor feed such as benzene, toluene, hexane, cyclohexane etc. It is necessary to know the composition of the feed to then calculate the average molecular weight of this stream to be able to calculate the correct hydrogen feed rate. The excess amount of hydrogen in the reaction mixture suppresses the coke formation which is believed to lead to catalyst deactivation.

### Hydrocracking Catalyst

The hydrocracking catalyst used in the process according to the invention comprises 0.01-1 wt % hydrogenation metal in relation to the total catalyst weight and a zeolite having a pore size of 5-8 Å and a silica (SiO2) to alumina (Al2O3) molar ratio of 5-200.

Catalysts having hydrocracking activity (“hydrocracking catalyst”) are described on pages 13-14 and 174 of Hydrocracking Science and Technology (1996) Ed. Julius Scherzer, A. J. Gruia, Pub. Taylor and Francis. Hydrocracking reactions proceed through a bifunctional mechanism which requires a relatively strong acid function, which provides for the cracking and isomerization and which provides breaking of the sulphur-carbon bonds comprised in the organic sulfur compounds comprised in the feed, and a metal function, which provides for the olefin hydrogenation and the formation of hydrogen sulfide. Many catalysts used for the hydrocracking process are formed by composting various transition metals with the solid support such as alumina, silica, alumina-silica, magnesia and zeolites.

Hydrocracking catalysts that are particularly suitable for the process of the present invention comprise a molecular sieve, preferably a zeolite, having a pore size of 5-8 Å.

Zeolites are well-known molecular sieves having a well-defined pore size. As used herein, the term “zeolite” or “aluminosilicate zeolite” relates to an aluminosilicate molecular sieve. An overview of their characteristics is for example provided by the chapter on Molecular Sieves in Kirk-Othmer Encyclopedia of Chemical Technology, Volume 16, p 811-853; in Atlas of Zeolite Framework Types, 5th edition, (Elsevier, 2001). Preferably, the hydrocracking catalyst comprises a medium pore size aluminosilicate zeolite or a large pore size aluminosilicate zeolite. Suitable zeolites include, but are not limited to, ZSM-5, MCM-22, ZSM-11, beta zeolite, EU-1 zeolite, zeolite Y, faujastite, ferrierite and mordenite. The term “medium pore zeolite” is commonly used in the field of zeolite catalysts. Accordingly, a medium pore size zeolite is a zeolite having a pore size of about 5-6 Å . Suitable medium pore size zeolites are 10-ring zeolites, i.e. the pore is formed by a ring consisting of 10 SiO₄ tetrahedra. Suitable large pore size zeolites have a pore size of about 6-8 Å and are of the 12-ring structure type. Zeolites of the 8-ring structure type are called small pore size zeolites. In the above cited Atlas of Zeolite Framework Types various zeolites are listed based on ring structure. Most preferably the zeolite is ZSM-5 zeolite, which is a well-known zeolite having MFI structure.

Preferably, the silica to alimuna ratio of the ZSM-5 zeolite is in the range of 20-200, more preferably in the range of 30-100.

The zeolite is in the hydrogen form: i.e. having at least a portion of the original cations associated therewith replaced by hydrogen. Methods to convert an aluminosilicate zeolite to the hydrogen form are well known in the art. A first method involves direct ion exchange employing an acid and/or salt. A second method involves base-exchange using ammonium salts followed by calcination.

Furthermore, the catalyst composition comprises a sufficient amount of hydrogenation metal to ensure that the catalyst has a relatively strong hydrogenation activity. Hydrogenation metals are well known in the art of petrochemical catalysts.

The catalyst composition preferably comprises 0.01-1 wt-% hydrogenation metal, more preferably 0.01-0.7 wt-%, most preferably 0.01-0.5 wt-% hydrogenation metal, more preferably 0.01-0.3 wt-%. The catalyst composition may more preferably comprise 0.01-0.1 wt-% or 0.02-0.09 wt-% hydrogenation metal. In the context of the present invention, the term “wt %” when relating to the metal content as comprised in a catalyst composition relates to the wt % (or “wt-%”) of said metal in relation to the weight of the total catalyst, including catalyst binders, fillers, diluents and the like. Preferably, the hydrogenation metal is at least one element selected from Group 10 of the periodic table of Elements. The preferred Group 10 element is platinum. Accordingly, the hydrocracking catalyst used in the process of the present invention comprises a zeolite having a pore size of 5-8 Å , a silica (SiO₂) to alumina (Al₂O₃) molar ratio of 5-200 and 0.01-1 wt-% platinum (in relation to the total catalyst)

The hydrocracking catalyst composition may further comprise a binder. Alumina (Al₂O₃) is a preferred binder. The catalyst composition of the present invention preferably comprises at least 10 wt-%, most preferably at least 20 wt-% binder and preferably comprises up to 40 wt-% binder. In one embodiment, the hydrogenation metal is deposited on the binder, which preferably is Al₂O₃.

According to one embodiment of the invention the hydrocracking catalyst is a mixture of the hydrogenation metal on a support of an amorphous alumina and the zeolite.

According to another embodiment of the invention the hydrocracking catalyst comprises the hydrogenation metal on a support of the zeolite. In this case, the hydrogenation metal and the zeolite giving cracking functions are in closer proximity to one another which translates into a shorter diffusion length between the two sites. This allows high space velocity, which translates into smaller reactor volumes and thus lower CAPEX. Accordingly, in some preferred embodiments, the hydrocracking catalyst is the hydrogenation metal on a support of the zeolite and step (b) is performed at a Weight Hourly Space Velocity of 0.1-15 h⁻¹.

### Hydrocracking Product Stream

The hydrocracking product stream comprises benzene and LPG. The hydrocracking product stream typically further comprises methane and hydrogen. The hydrocracking product stream may further comprise toluene and xylene, mainly because not all of the monoaromatic compounds are alkylated in step (a).

In some preferred embodiments, the process according to the invention comprises the step of separating the hydrocracking product stream into benzene and LPG and the rest of the hydrocracking product stream. The rest of the hydrocracking product stream may comprise a stream comprising alkyl monoaromatic compounds, in particular toluene and xylene.

The stream comprising the alkyl monoaromatic compounds separated from the hydrocracking product stream may be fed back to the alkylation reactor. This has the advantage that the alkyl monoaromatic compounds which were not alkylated during step (a) and which were not converted into benzene are again subjected to alkylation. This will result in that a higher amount of the alkyl monoaromatic compounds is converted into benzene. Eventually, all alkyl monoaromatic compounds in the feed stream may be converted into benzene and LPG.

### Separation of Benzene and LPG

As described above, the process according to the invention preferably further comprises the step of separating the hydrocracking product stream into benzene, LPG and optionally a stream comprising alkyl monoaromatic compounds, typically toluene and xylene.

Preferably, the stream comprising the alkyl monoaromatic compounds are fed back to the alkylation reactor. Accordingly, in some preferred embodiments of the process of the invention, the source feed stream comprises the stream comprising the alkyl monoaromatic compounds separated from the hydrocracking product stream and fed back to the alkylation reactor.

The hydrocracking product stream may be subjected to separation by standard means and methods suitable for separating methane and unreacted hydrogen comprised in the hydrocracking product stream as a first separate stream, the LPG comprised in the hydrocracking product stream as a first separate stream and the BTX as a second separate stream. Preferably, the BTX is separated from the hydrocracking product stream by gas-liquid separation or distillation. One non-limiting example of such a separation method includes a series of distillation steps. The first distillation step, at moderate, temperature is to separate most of the aromatic species (liquid product) from the hydrogen, H₂S, methane and LPG species. The gaseous stream from this distillation is further cooled (to about −30° C.) and distilled again to separate the remaining aromatics species and most of the propane and butane. The gaseous product (mainly hydrogen, H₂S, methane and ethane) is then further cooled (to about −100° C.) to separate the ethane and leave the hydrogen, H₂S and methane in the gaseous stream that will be recycled to the reactor. To control the levels of H₂S and methane in the reactor feed, a proportion of recycle gas stream is removed from the system as a purge. The quantity of material that is purged depends on the levels of methane and H₂S in the recycle stream which in-turn depend on the feed composition. The purge stream will have the same composition as the recycle stream. As the purge will contain mainly hydrogen and methane it is suitable for use as a fuel gas or may be further treated (e.g. via a pressure swing adsorption unit) to separately recover a high purity hydrogen stream and a methane/H₂S stream which can be used as a fuel gas.

Preferably, after the BTX is separated from the hydrocracking product stream, toluene and xylene are separated from benzene by gas-liquid separation or distillation.

### Second Feed Stream

Preferably, step (b) of the process according to the invention involves further feeding in the hydrocracking reactor a second feed stream comprising C5-C12 hydrocarbons.

Preferably, the second feed stream comprises pyrolysis gasoline, straight run naphtha, light coker naphtha and coke oven light oil or mixtures thereof.

In some preferred embodiments, step (b) of the process according to the invention involves further feeding in the hydrocracking reactor a second feed stream comprising C5-C12 hydrocarbons and the source feedstream is the stream comprising the alkyl monoaromatic compounds separated from the hydrocracking product stream fed back to the alkylation reactor. In these embodiments, the second feed stream is first hydrocracked and the stream comprising the alkyl monoaromatic compounds separated from the hydrocracking product stream is fed to the alkylation reactor as the source stream.

### Process System

Preferably, the process according to the invention is carried out in a system in which an inlet of the alkylation reactor is heated by an outlet of the hydrocracking reactor. An advantage of this system is that a more energy efficient process is obtained. The hydrocracking reactor is operated at a temperature that is in the same range as the temperature in the alkylation reactor. The hydrocracking reaction is exothermic while the alkylation reaction is endothermic. According to the system of the invention, the heat generated during hydrocracking is advantageously used for heating of the alkylation reactor.

Although the invention has been described in detail for purposes of illustration, it is understood that such detail is solely for that purpose and variations can be made therein by those skilled in the art without departing from the spirit and scope of the invention as defined in the claims.

It is further noted that the invention relates to all possible combinations of features described herein, preferred in particular are those combinations of features that are present in the claims.

It is noted that the term “comprising” does not exclude the presence of other elements. However, it is also to be understood that a description on a product comprising certain components also discloses a product consisting of these components. Similarly, it is also to be understood that a description on a process comprising certain steps also discloses a process consisting of these steps.

The present invention will now be elucidated by the following experiment.

### EXPERIMENT 1

A feed stream of xylene was fed to an alkylation reactor having an alkylation catalyst comprising Cs-13X zeolite molecular sieve and Cobaltborate. Methanol was also fed to the reactor. The molar ratio between xylene to methanol was 60% to 40%. The temperatures for the reaction are mentioned in Table 1. The pressure was 1 atm and WHSV was 4-5 h⁻¹.

The proportions of the obtained compounds are summarized in Table 1 (in wt %).

TABLE 1

| | Ethyl | | Methyl | Ethyl | | | | |
|---|---|---|---|---|---|---|---|---|
| Temp | methyl | Diethyl | vinyl | vinyl | Divinyl | | | |
| (° C.) | benzene | benzene | benzene | benzene | benzene | Naphthalene | Xylene | Methanol |
| 400 | 10-20 | 5-10 | 5-10 | 5-10 | 2-5 | — | 50-55 | 15-20 |
| 430 | 10-20 | 5-10 | 5-10 | 5-10 | 2-5 | — | 45-50 | 10-15 |
| 450 | 10-20 | 5-10 | — | 5-10 | 2-5 | 2-5 | 45-50 | 10-15 |

When the reaction temperature was 450° C., some portion of naphthalene was formed, which is not desirable. Hence, the reaction temperature is preferably below 450° C., for example at most 440° C.

About 10-15% of xylene in the feed stream was alkylated to result in various compounds mentioned in Table 1. The methyl groups of xylene were converted into ethyl group or vinyl group, or remained as methyl group.

Alkylation product streams were thus obtained enriched in monoaromatic compounds having ethyl group or vinyl group instead of methyl group. The alkylation product stream can be subjected to a hydrocracking step as defined in step (b) of the process of the invention.

Diethyl benzene, ethyl vinyl benzene and divinyl benzene will be converted into benzene in the subsequent hydrocracking step. Hence, it can be concluded that xylene which would not be converted into benzene by hydrocracking can increase benzene yield by first subjecting it to alkylation.

Ethyl methyl benzene and methyl vinyl benzene will be converted into toluene. If toluene is fed back to the alkylation reactor, some of the toluene will be converted into ethylbenzene. Ethylbenzene will be converted into benzene by subsequent hydrocracking.

1. A process for producing benzene and LPG comprising the steps of:
(a) reacting a source feed stream comprising monoaromatic compounds of formula (I), 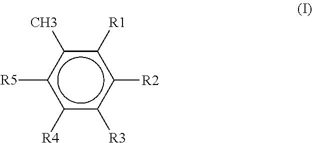
wherein R1-R5 are the same or different and are chosen from hydrogen or a linear alkyl group of 1-10 carbon atoms,
and methanol in an alkylation reactor comprising a basic catalyst to obtain an alkylation product stream and subsequently
(b) contacting the alkylation product stream in the presence of hydrogen in a hydrocracking reactor with a hydrocracking catalyst comprising 0.01-1 wt-% hydrogenation metal in relation to the total catalyst weight and a zeolite having a pore size of 5-8 Å and a silica (SiO2) to alumina (Al2O3) molar ratio of 5-200 to produce a hydrocracking product stream comprising benzene and LPG under process conditions including a temperature of 425-580° C., a pressure of 300-5000 kPa gauge and a Weight Hourly Space Velocity of 0.1-15 ⁻¹.

2. The process according to claim 1, wherein step (b) involves further feeding in the hydrocracking reactor a second feed stream comprising C5-C12 hydrocarbons.

3. The process according to claim 1, wherein the monoaromatic compounds comprise toluene, xylene and/or 1,3,5-trimethyl benzene.

4. The process according to claim 1, wherein the source feed stream comprises C5-C12 hydrocarbons.

5. The process according to claim 1, wherein the second feed stream comprises pyrolysis gasoline, straight run naphtha, light coker naphtha and coke oven light oil or mixtures thereof.

6. The process according to claim 1, further comprising the step of separating the hydrocracking product stream into benzene, LPG and optionally a stream comprising alkyl monoaromatic compounds.

7. The process according to claim 6, wherein the source feed stream comprises the stream comprising the alkyl monoaromatic compounds separated from the hydrocracking product stream and fed back to the alkylation reactor.

8. The process according to claim 1, wherein the alkylation reactor comprises an X or Y zeolite catalyst modified with B, P, Na, K, Cs or Rb or mixtures thereof.

9. The process according to claim 1, wherein in the alkylation reactor the ratio between methanol and the monoaromatic compound is between 1:1 and 1:20.

10. The process according to claim 1, wherein the temperature in the alkylation reactor is between 100 and 1000° C.

11. The process according to claim 1, wherein the temperature in the alkylation reactor is between 350 and 440° C.

12. The process according to claim 1, wherein the process is carried out in a system in which an inlet of the alkylation reactor is heated by an outlet of the hydrocracking reactor.

13. The process according to claim 4, wherein the source feed stream comprises pyrolysis gasoline, straight run naphtha, light coker naphtha and coke oven light oil or mixtures thereof
